(19) United States
(12) Patent Application Publication
(10) Pub. No.: US 2022/0073910 A1
(43) Pub. Date: Mar. 10, 2022

(54) A Cross-Linking Approach to Map Small Molecule-RNA Binding Sites in Cells

(71) Applicant: The Scripps Research Institute, La Jolla, CA (US)

(72) Inventor: Matthew D. Disney, Jupiter, FL (US)

(21) Appl. No.: 17/419,654

(22) PCT Filed: Feb. 11, 2020
(86) PCT No.: PCT/US2020/017725

§ 371 Date: Jun. 29, 2021

Related U.S. Application Data
(60) Provisional application No. 62/804,308, filed on Feb. 12, 2019.

(51) Int. Cl. C12N 15/10; C12Q 1/6851; C12Q 1/6886

(52) U.S. Cl. CPC C12N 15/1096; C12Q 1/6851; C12Q 2600/178; C12Q 2600/158; C12Q 1/6886

(57) ABSTRACT

Disclosed herein are compounds and methods to identify the direct RNA targets of small molecules in cells is described. The approach, dubbed Chemical Cross-Linking and Isolation by Pull-down to Map Small Molecule-RNA Binding Sites (Chem-CLIP-Map-Seq), appends a cross-linker and a purification tag onto a small molecule. In cells, the compound binds to RNA and undergoes a proximity-based reaction. The cross-linked RNA is purified and then amplified using a universal reverse transcription (RT) primer and gene-specific PCR primers. At nucleotides proximal to the binding site, RT “stops” are observed. This approach has broad utility in identifying and validating the RNA targets and binding sites of small molecules in the context of a complex cellular system.

### CROSS-REFERENCE TO RELATED APPLICATION

This application claims the priority of U.S. provisional application Ser. No. 62/804,308, filed Feb. 12, 2019, the disclosure of which is incorporated herein by reference in its entirety.

### STATEMENT OF GOVERNMENT SUPPORT

This invention was made with government support under grant number GM097455 awarded by the National Institutes of Health. The government has certain rights in the invention.

### BACKGROUND

As aberrant expression and mutation of human RNAs are linked to diseases, RNA is a desirable target for small molecule therapeutics.^{1, 2} Although it is difficult to drug RNA, salient examples of success include compounds that target bacterial riboswitches³ and human pre-mRNAs,^{4, 5} identified from phenotypic screens. There are even fewer examples of the purposeful design of small molecules that directly engage RNA targets and modulate biological activity in cells, largely for RNA repeat expansions and microRNA (miRNA) precursors.⁶ As the RNA targeting area continues to evolve and develop, there is a need for approaches to define the bona fide targets of RNA-directed small molecules in intact living systems, to establish modes of action, and to facilitate development of improved chemical probes that could emerge into medicines.

Indeed, these types of methods have been well established for protein drug targets. For example, covalent approaches, such as Cross-Linking and ImmunoPrecipitation (CLIP), have been used to characterize RNA-protein complexes.⁷ In CLIP, proteins are cross-linked to their bound RNAs by irradiation with light. These studies have transformed the understanding of how proteins regulate RNA including their binding site localization and the exclusion and inclusion of exons. Activity-based protein profiling (ABPP) and related approaches can assign enzymatic activity across the proteome and elucidate “ligand-ability” on a proteome-wide scale.^{8, 9}

Recently, covalent approaches for small molecule recognition of nucleic acids have been described. For example, by using derivatives of cis-platin, it was found that this important anti-cancer compound recognizes the ribosome and affects its activity.¹⁰ Modular approaches have also been developed to probe the RNA targets of designer small molecules, dubbed Chemical Cross-Linking and Isolation by Pull-down (Chem-CLIP).^{11,12} In Chem-CLIP, a chemical cross linker and biotin purification tag are appended to an RNA-binding small molecule (FIG. 1). When the small molecule binds an RNA, the cross linker is brought into high effective concentration and undergoes a proximity-based reaction.^{11,12} The covalent adducts can be purified from cells via biotin to identify the small molecule's cellular targets. In previous studies, this approach has been used, for example, to map ligand binding sites onto repeating transcripts.¹³ In that application, total RNA was incubated with an antisense oligonucleotide complementary to the target of interest and RNase H, digesting the RNA target into fragments. Fragments that reacted with the Chem-CLIP probe were isolated with streptavidin beads and analyzed by RT-qPCR with a set of gene-specific primers.¹³

### SUMMARY

Provided herein are methods of mapping an RNA binding site of an RNA-binding small molecule, the binding site being disposed within an RNA sequence library, comprising

contacting the RNA of the sequence library and an effective amount of a compound, the compound comprising a conjugate of the RNA-binding small molecule and an RNA cross-linking module and a purification module, such that the RNA cross-linking module can react with the RNA associated with the RNA binding site of the small molecule; then,

purifying the compound and an RNA target cross-linked by the cross-linking module using affinity of the purification module and a complementary immobilized reagent; then,

reverse transcribe the RNA with a primer to create cDNA from the purified RNA bound and thereby cross-linked to the small molecule; then,

amplify the cDNA using a primer set suitable for RT-qPCR or high throughput sequencing (RNA-seq) then,

analyze the cDNA by RNA-seq or RT-qPCR to identify the target, as determined by the enrichment in the purified fractions; then

analyze the cDNA to identify reverse transcriptase sequence stops; then,

map the sequence stops onto the sequence or secondary structure of the RNA of the sequence library;

to identify the binding sites of the RNA-binding small molecule within the RNA library.

In various embodiments, the RNA cross-linking module can comprise a N,N-bis(2-chloroethyl)aniline (chlorambucil) group. In various embodiments, the purification module can comprise a biotin group.

More specifically, provided are methods wherein the compound comprising a conjugate of the RNA-binding small molecule and an RNA cross-linking module and a purification module is of formula 2. Further, the compound can comprise an N-methyl-piperazinyl-bis-benzimidazole group as an RNA-binding moiety. In various embodiments, the compound can be Targaprimir-96 (compound 1).

Further, in various embodiments the above-described method can comprise precisely targeting cellular inactivation of an oncogenic non-coding RNA precursor via cross-linking, comprising contacting a cell expressing the non-coding RNA precursor and an effective amount of the compound comprising a conjugate of the RNA-binding small molecule and an RNA cross-linking module and a purification module. More specifically, the oncogenic non-coding RNA precursor can comprise oncogenic primary microRNA-96 (pri-miR-96). In various embodiments, the conjugate is a cross-linking conjugate of Targaprimir-96 and chlorambucil.

Further, in various embodiments the above-described method can comprise enhancing expression of FOXO1 protein in breast cancer cells, comprising contacting the cells with an effective amount of the compound comprising a conjugate of the RNA-binding small molecule and an RNA cross-linking module and optionally a purification module. More specifically, the breast cancer cells can be present in a human patient. More specifically, the compound can be a covalent conjugate comprising Targaprimir-96 and chlorambucil.

Further, in various embodiments the above-described method can comprise triggering apoptosis in triple negative breast cancer cells, comprising contacting the cells with an effective amount of the compound comprising a conjugate of the RNA-binding small molecule and an RNA cross-linking module and a purification module. The breast cancer cells can be present in a human patient. The compound can be a covalent conjugate of Targaprimir-96 and chlorambucil.

Further provided are methods of treating triple negative breast cancer, comprising administering to a patient afflicted therewith an effective dose of the compound comprising a conjugate of the RNA-binding small molecule and an RNA cross-linking module and a purification module. The breast cancer can comprise expression of oncogenic primary microRNA-96 (pri-miR-96). The compound can be a covalent conjugate of Targaprimir-96 and chlorambucil.

In various embodiments, the RNA sequence library can comprise a transcriptome. The transcriptome can be viral, or the transcriptome can be mammalian, or the transcriptome can be bacterial.

In various embodiments, the RNA sequence library can comprise one or more of synthetic, semi-synthetic, or natural RNA. The RNA sequence library can comprise the genome of an RNA virus.

In various embodiments, the method can be carried out in vitro, or can be carried out in living cells, wherein the cells can be virally- or bacterially-infected cells; or the method can be carried out in preclinical animal models.

Further provided is, in various embodiments, the above-described method wherein a set of RNA sequences and a set of candidate RNA-binding small molecules are assayed in a 2-dimensional parallel array.

Further provided is, in various embodiments, a conjugate of an RNA-cross-linking moiety and a RNA-binding small molecule. More specifically, in the conjugate, the RNA-cross-linking moiety can comprise an N,N-bis(2-chloroethyl)aniline (chlorambucil) group. Further, in the conjugate, the RNA-binding small molecule can comprise an N-methyl-piperazinyl-bis-benzimidazole group. For example, the conjugate can comprise Targaprimir-96.

### BRIEF DESCRIPTION OF THE FIGURES

FIG. 1 shows the secondary structure of pri-miR-96 with small molecule binding sites indicated (top) and the miR-96-FOXO1 pathway (bottom).

FIG. 2 shows the chemical structures of the compound used in this study. Compound 1 is parent compound “Targaprimir-96”; compound 2 is the Targaprimir-96 Chem-CLIP probe; and compound 3 is a control compound containing reactive and purification modules but lacking RNA-binding modules.

FIG. 3 shows the effects of compounds 1, 2, and 3 in MDA-MB-231 TNBC and MCF-10A (nontumorigenic mammary epithelial breast) cells. A—Effects of compounds 1 and 2 (50 nM) on mature miR-96 and pri-miR-96 levels. B—Effects of compounds 1 and 2 (50 nM) on FOXO1 protein expression. C— Profiling of miRNAs predicted to target FOXO1 mRNA by TargetScan in compound 2-treated MDA-MB-231 cells. D—Volcano plot profiling the effect of compound 2 on all miRNAs that are expressed in MDA-MB-231 cells (blue circles are miRNAs predicted to target FOXO1 mRNA by TargetScan). E—Effects of compounds 1, 2, and 3 on Caspase 3/7 activity, indicative of apoptosis, in MDA-MB-231 TNBC cells. F—Effects of compounds 1, 2, and 3 on Caspase 3/7 activity, indicative of apoptosis, in MCF-10A normal breast epithelial cells.

FIG. 4 shows the mapping the binding sites of compound 2 within pri-miR-96 in MDA-MB-231 TNBC cells. A—Scheme of amplification approach to identify small molecule binding site in pri-miR-96. B— In cellulis mapping of the binding site for compound 2 within pri-miR-96 in MDA-MB-231 TNBC cells. Percentages indicate the number of sequences observed terminating at the respective nucleotide adjacent to the percentage.

### DETAILED DESCRIPTION

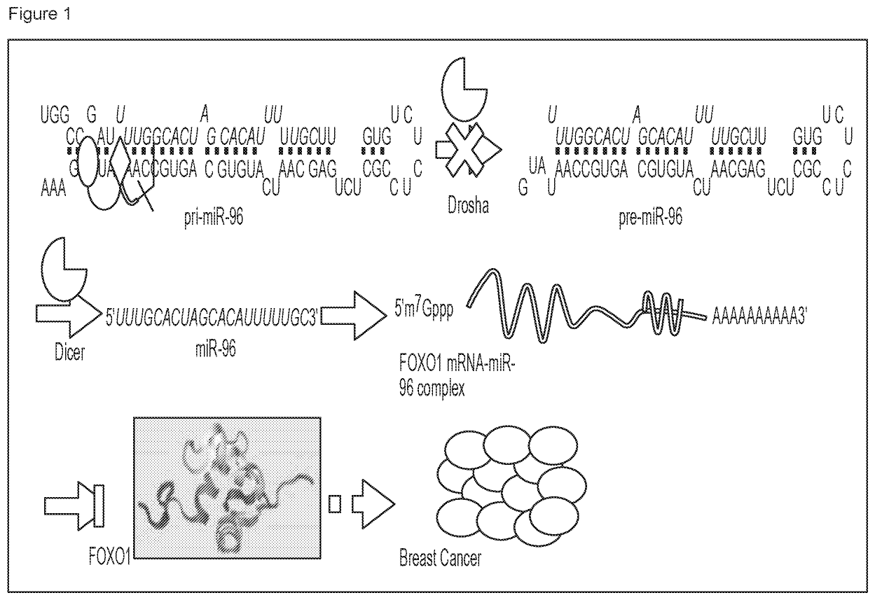

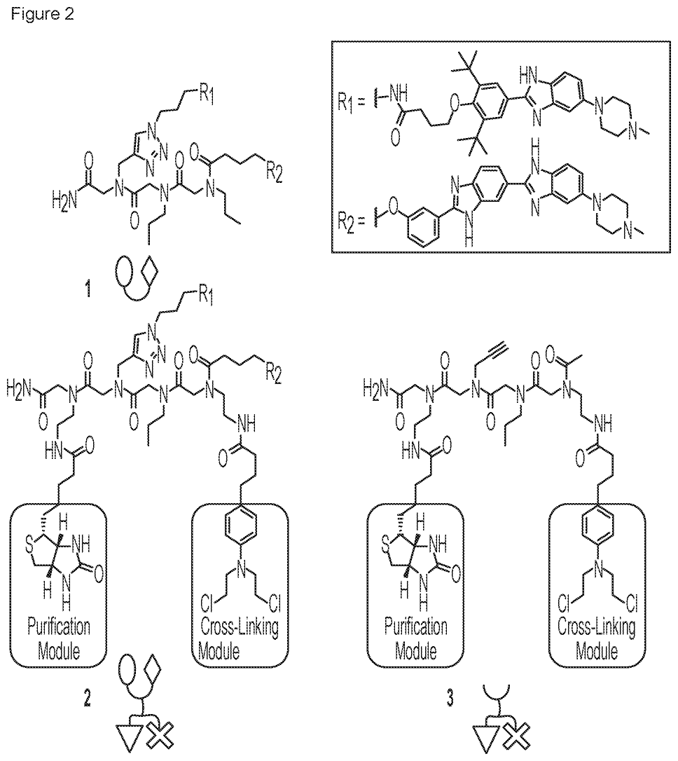

RNA repeat expansions, however, are atypical targets of small molecules. We therefore sought to develop a Chem-CLIP-based mapping approach for RNAs with more diverse composites of structures such as microRNA (miRNA) precursors, named Chem-CLIP-Map-Seq. To do so, we used the primary transcript of oncogenic microRNA-96 (pri-miR-96) as a test case (FIG. 1). The small molecule Targaprimir-96 (1) binds selectively to the Drosha processing site of pri-miR-96 and selectively inhibits its biogenesis triple negative breast cancer (TNBC) cells and an in vivo xenograft model.¹⁴ The Chem-CLIP probe for Targaprimir-96 (2) and a derivative of 2 lacking the RNA-binding modules (control; 3) (FIG. 2) were synthesized and studied in MDA-MB-231 TNBC cells.

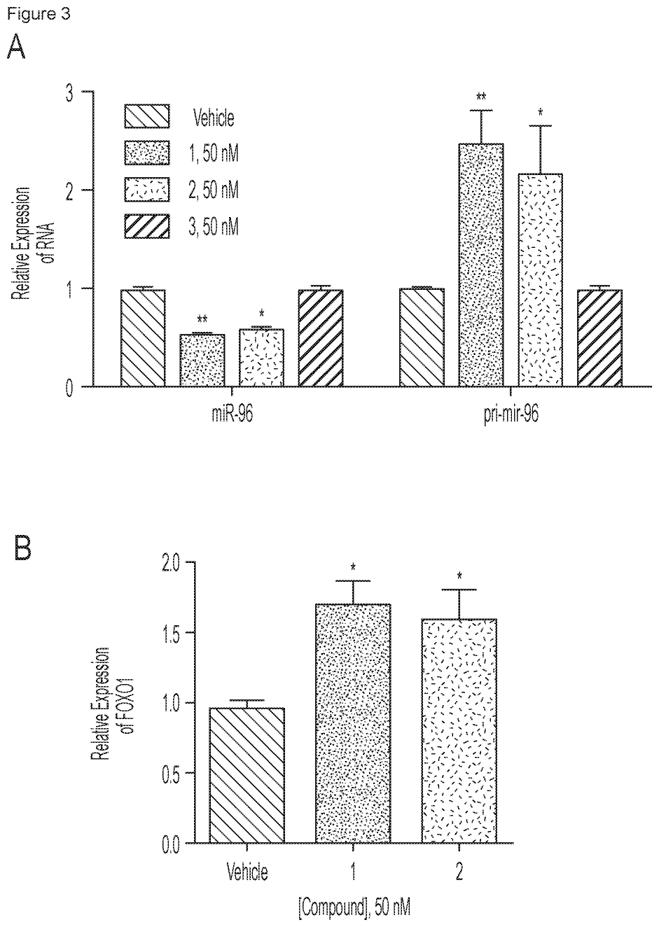

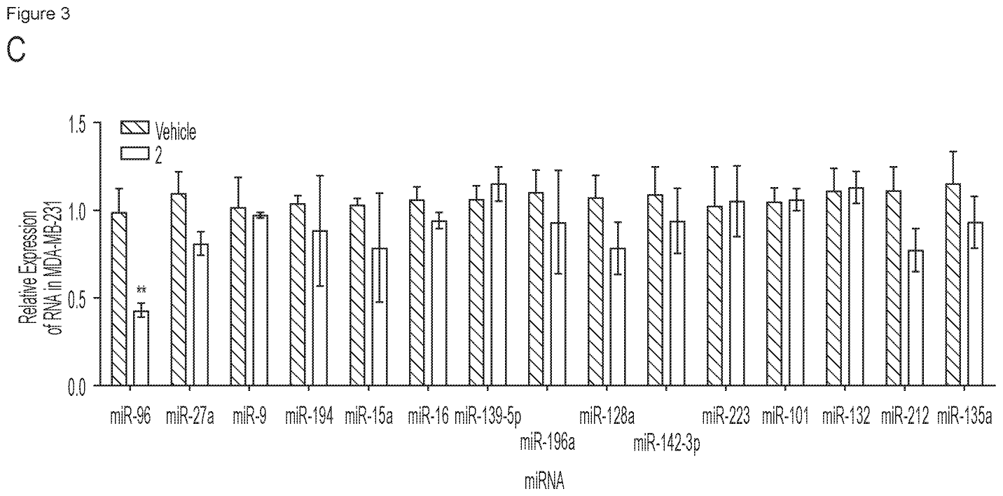

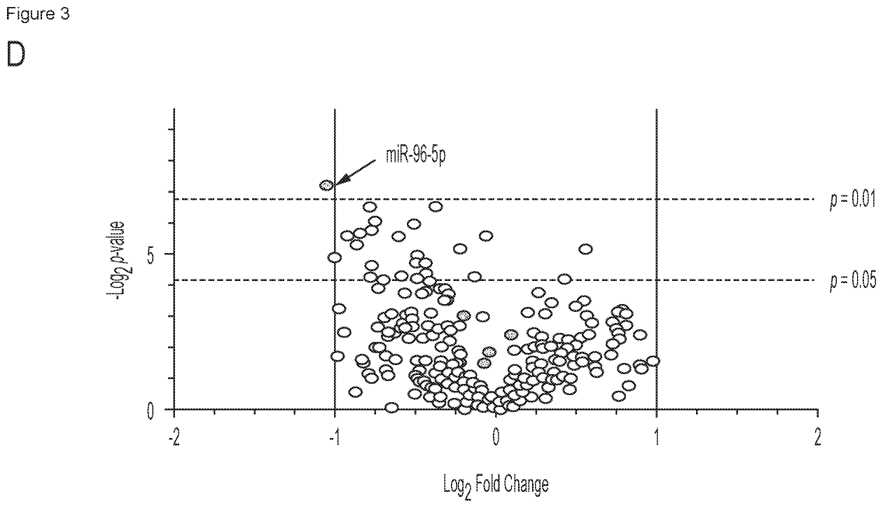

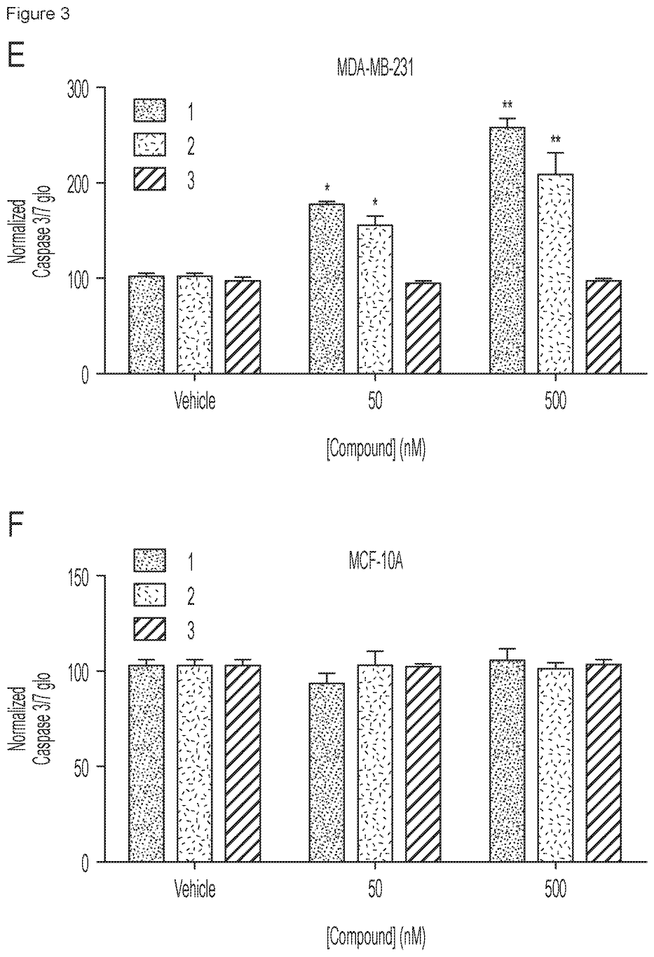

When MDA-MB-231 cells were treated with the Chem-CLIP probe 2 (50 nM), capable of cross-linking to pri-miR-96, mature miR-96 levels were reduced to a similar extent as the parent compound 1 (FIG. 3A). No effect was observed with control compound 3 (FIG. 3A). To confirm that both 1 and 2 reduced mature miR-96 levels by inhibiting Drosha processing, we measured pri-miR-96 levels by RT-qPCR, the results of which show that both compounds increased abundance of this RNA by ca. 1.5-fold at 50 nM (FIG. 3A).

Since miR-96 suppresses apoptosis in cancer cells by repressing the translation of Forkhead box 01 (FOXO1) transcription factor,¹⁵ the effects of 1, 2, and 3 on FOXO1 protein levels were measured in MDA-MB-231 cells. As expected, based on RT-qPCR analysis, only 1 and 2, but not 3, enhanced the expression of FOXO1 protein (FIG. 3B). To validate that the increase in FOXO1 levels was mediated by inhibition of miR-96, we studied the effects of 1 and 2 on all 15 miRNAs predicted to target the FOXO1 3′ untranslated region (UTR) by TargetScan (FIG. 3C). Only miR-96 was inhibited significantly, supporting that the increase in FOXO1 protein expression is due to 1's and 2's inhibitory effect on miR-96 biogenesis (FIG. 3C). To further probe 2's selectivity for pri-miR-96, we performed an unbiased profiling experiment on 373 miRNAs expressed in MDA-MB-231 TNBC cells (based on Qiagen miRNA Human miRbase profiler HC). As shown in the volcano plot in FIG. 3D, miR-96 was the most affected, indicating the selectivity of covalent compound 2.

We next studied the ability of 1 and 2 to trigger apoptosis, i.e., reverse phenotype, in MDA-MB-231 by measuring Caspase activity. Analogous experiments were completed in healthy breast epithelial MCF-10A cells that do not aberrantly express miR-96. Both 1- and 2-treated MDA-MB-231 cells had increased Caspase activity indicative of apoptosis (FIG. 3E). Compound 3 had no effect on Caspase activity, as expected. Importantly, neither 1 nor 2 had any effect on MCF-10A cells (FIG. 3F). Thus, the covalent compounds show relative selectivity for cancerous over healthy breast cells.

We previously studied the engagement of pri-miR-96 in vitro by incubating radioactively labeled pri-miR-96 RNA with 2 and then pulling down 2-pri-miR-96 adducts with streptavidin beads.¹⁴ Specific engagement of pri-miR-96 by 2 was observed as and no pulldown was observed with 3 which lacks RNA-binding modules.¹⁴ To further show that the RNA-binding modules drive recognition of the pri-miR-96 target, we previously completed an in vitro competitive (C-)Chem CLIP experiment in which pri-miR-96 was incubated with a constant concentration of 2 (1000 nM) and increasing amounts of 1. Indeed, a 1-dependent decrease in the amount of RNA pulled down by 2 was observed.

To the study the engagement of pri-miR-96 by 2 in cells, we previously pulled down the RNAs with which 2 reacted with streptavidin beads and quantified pri-miR-96's enrichment via RT-qPCR.¹⁴ Indeed, a 5-fold enrichment of pri-miRNA-96 was observed in the pulled down fraction (as compared to the amount present in the sample prior to incubation with streptavidin beads). Akin to in vitro studies, we completed a cellular C-Chem-CLIP study to show that the RNA-binding module is required for reaction. As expected for a selective interaction, the amount of pri-miR-96 pulled down decreased as a function of the concentration of 1.¹⁴

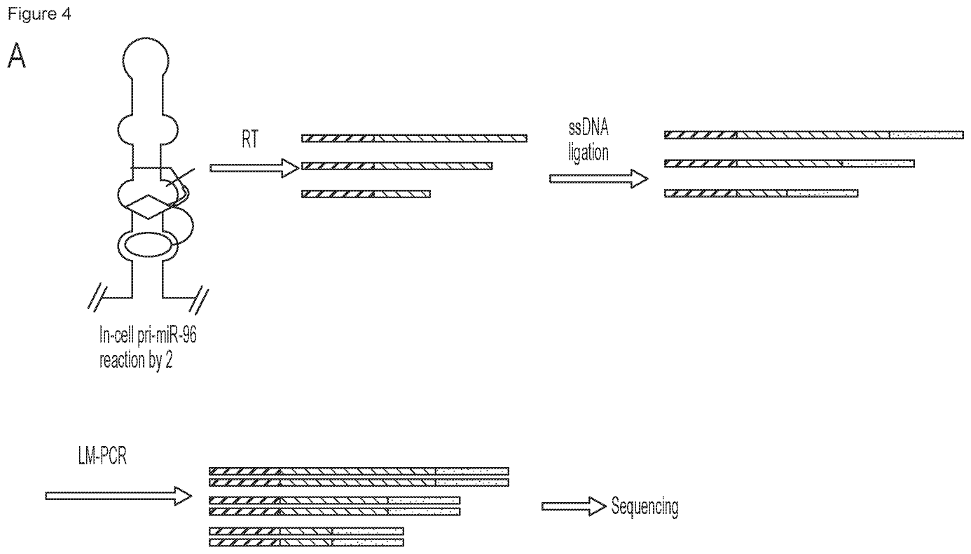

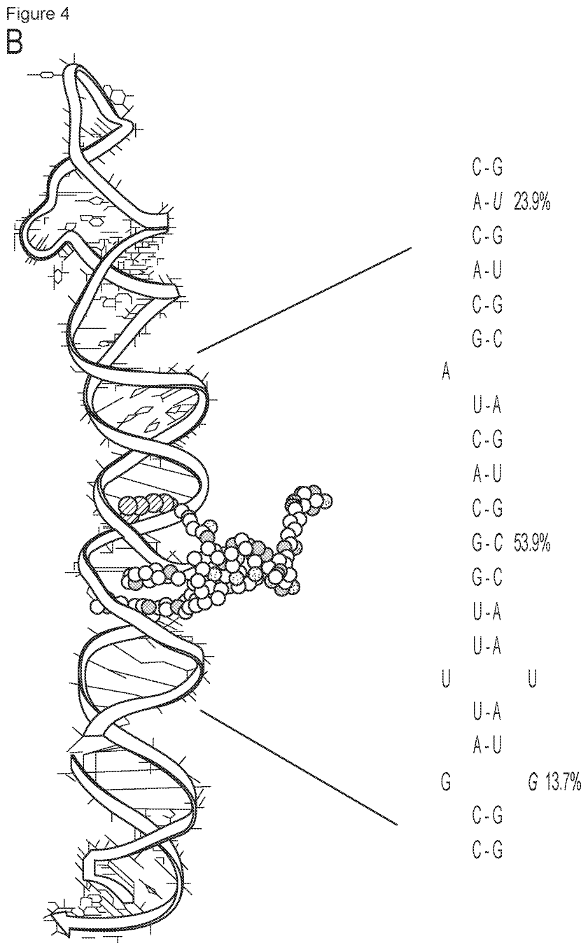

Given the enrichment of pri-miR-96 in the pulled down fraction and 2's pro-apoptotic properties, we mapped 2's binding site within pri-miR-96 in cells. We defined 2's cellular binding site within pri-miR-96. [Please note that for these experiments, a significantly higher concentration of 2 was required to afford sufficient material for analysis, 1000 nM or 20 times higher than that required to inhibit miR-96 biogenesis and trigger apoptosis.] After reverse transcription, ssDNA adaptors were ligated to the cDNA, followed by which was cloned into NEB's pminiT 2.0 vector and sequenced. A schematic for this is shown in FIG. 4A. Mirroring the predicted and validated (via nuclease mapping¹⁴) binding site, 2's binding site was localized to nucleotides that are proximal to pri-miR-96's Drosha site (FIG. 4B). In particular, the most occupied binding site observed is a C residue four nucleotides upstream of the UU internal loop bound by 1 (54%; highlighted in red in FIG. 4). Two other reactions sites were observed: (i) a G residue in the GG internal loop that binds 1 (14%); and (ii) a U residue 13 nucleotides upstream of the UU internal loop that is part of the Drosha processing site (24%). To support our cellular mapping experiments, we modeled 2 bound to pri-miR-96 (FIG. 4B). Indeed, the distance between 2's cross-linking module and the most reacted nucleotides match precisely.

We have developed a cleavage-based approach named small molecule nucleic acid profiling by cleavage applied to RNA, or RiboSNAP, that is complementary to to Chem-CLIP-Map-Seq.¹³ In RiboSNAP, an RNA-binding small molecule is conjugated to bleomycin A5. Conjugation of the RNA binder to bleomycin A5 attenuates its ability to bind and cleave DNA, directing it towards the desired RNA target. RiboSNAP was used to identify the binding site of 1 within pri-miR-96, or RiboSNAP-Map.¹⁶ As expected, both Chem-CLIP-Map-Seq and RiboSNAP-Map localized the binding site of 1 to pri-miR-96's Drosha site.¹⁶ Collectively, these studies demonstrate that Chem-CLIP-Map-Seq is a powerful method to investigate target engagement in cells as well as define small molecule binding sites within a cellular RNA target and can be used synergistically.

Accordingly, in various embodiments, the disclosure provides compounds having (1) a RNA cross-linking module and (2) an RNA-binding module. In some cases, the RNA cross-linking module comprises an N,N-bis(2-chloroethylaniline (chlorambucil) group. In some cases, the RNA-binding module comprises an N-methyl-piperazinyl-bis-benzimidazole group. In some cases, the RNA-binding module comprises compound 1:

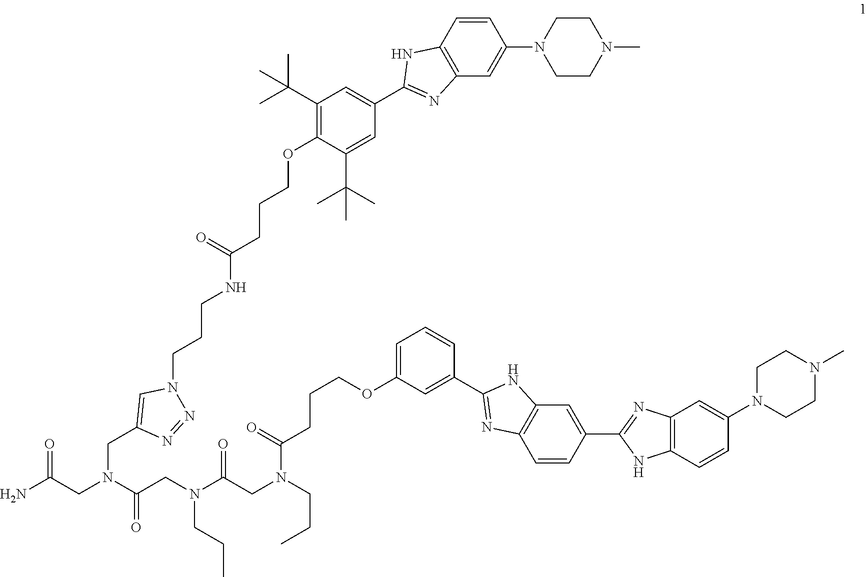 [SMILES: CCCN(CC(=O)N(CCC)CC(=O)N(CC(N)=O)Cc1cn(CCCNC(=O)CCCOc2c(C(C)(C)C)cc(-c3nc4cc(N5CCN(C)CC5)ccc4[nH]3)cc2C(C)(C)C)nn1)C(=O)CCCOc1cccc(-c2nc3ccc(-c4nc5cc(N6CCN(C)CC6)ccc5[nH]4)cc3[nH]2)c1]

known as Targaprimir-96).

The RNA cross-linking and/or purification module can be appended to the RNA-binding module via any available function group. For example, a nitrogen atom of an amide bond can be modified to include the RNA cross-linking module and/or purification module—as shown for compound 2 below:

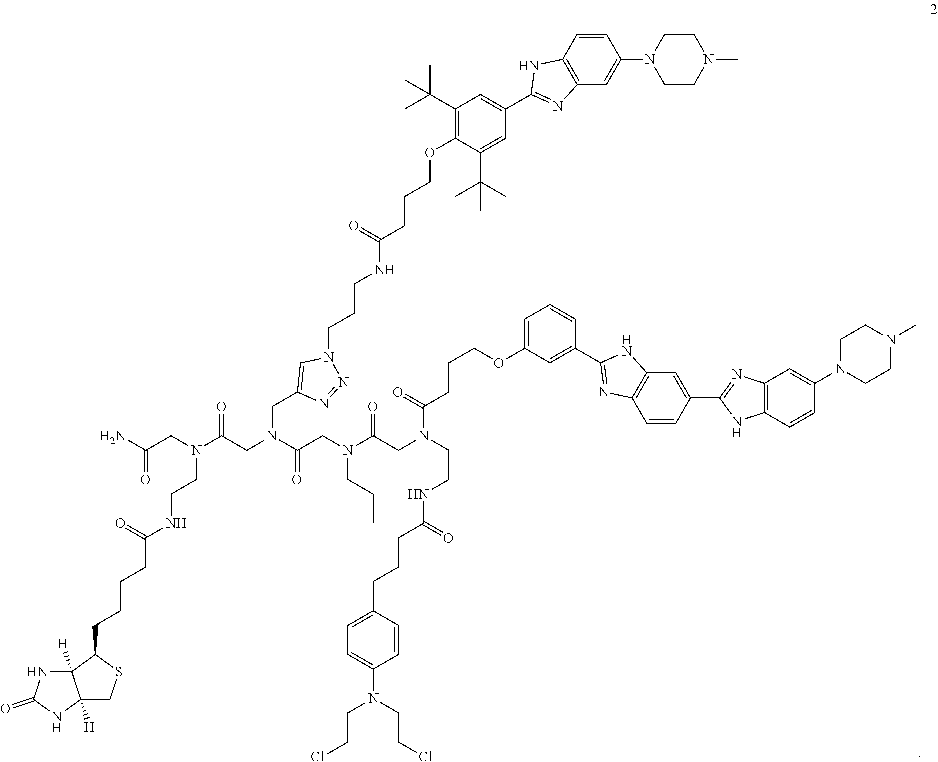 [SMILES: CCCN(CC(=O)N(CC(=O)N(CCNC(=O)CCCC[C@H]1SC[C@H]2NC(=O)N[C@H]21)CC(N)=O)Cc1cn(CCCNC(=O)CCCOc2c(C(C)(C)C)cc(-c3nc4cc(N5CCN(C)CC5)ccc4[nH]3)cc2C(C)(C)C)nn1)C(=O)CN(CCNC(=O)CCCc1ccc(N(CCCl)CCCl)cc1)C(=O)CCCOc1cccc(-c2nc3ccc(-c4nc5cc(N6CCN(C)CC6)ccc5[nH]4)cc3[nH]2)c1]

The purification module can comprise biotin, and a streptavidin based purification technique can be employed.

A comparator test compound which has a purification module and a RNA cross-linking module but no RNA binding module is shown in compound 3:

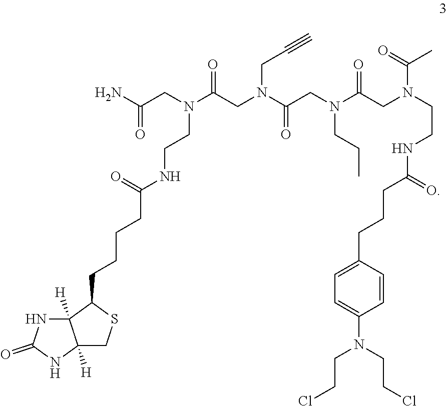 [SMILES: C#CCN(CC(=O)N(CCNC(=O)CCCC[C@H]1SC[C@H]2NC(=O)N[C@H]21)CC(N)=O)C(=O)CN(CCC)C(=O)CN(CCNC(=O)CCCc1ccc(N(CCCl)CCCl)cc1)C(C)=O]

The compounds disclosed herein can form a covalent complex with RNA via covalent bond formation between the RNA cross-linking module and the RNA. Accordingly, in various embodiments the disclosure provides a covalently-linked complex of compound 2 and RNA. In some embodiments, the disclosure provides a covalently linked complex of RNA and compound 2 without the biotin module.

In some embodiments, the crosslinking group can comprise an alkylating group, e.g., the (chloroethyl)amino group, a “nitrogen-mustard” group, of chlorambucil.

Also provided herein are methods of identifying RNA binding site of an RNA-binding small molecule, the binding site being disposed within an RNA sequence library, comprising (a) contacting the RNA of the sequence library and a compound comprising (i) the RNA-binding small molecule, (ii) an RNA cross-linking module, and (iii) a purification module, such that the RNA cross-linking module reacts with RNA when associated with the RNA binding site of the small molecule to form a complex; (b) purifying the complex via affinity purification; and (c); identifying the binding sites of the RNA-binding small molecule within the RNA library. In some cases, identifying the binding sites of the RNA-binding small molecule within the RNA library comprises (a) reverse transcribing the bound RNA with a primer to create cDNA from the purified bound RNA of the complex; (b) amplifying the cDNA using a primer set suitable for RT-qPCR or high throughput sequencing (RNA-seq); (c) analyzing the cDNA by RNA-seq or RT-qPCR to identify the targets, as determined by the enrichment in the purified fractions as compared to the starting cell lysate; (d) analyzing the cDNA to identify reverse transcriptase sequence stops; and (e) mapping the sequence stops onto the sequence or secondary structure of the RNA of the sequence library.

Formation of the covalently-linked complex of the compound with RNA provides, in various embodiments, a method of identifying an RNA, comprising contacting the RNA with a compound disclosed herein, wherein identifying comprises reverse transcribing the RNA with a primer to create cDNA, amplifying the cDNA using a suitable primer (e.g., a primer suitable for RT-qPCR or high-throughput sequencing, RNA-seq), analyzing the cDNA to identify the targets (e.g., by RT-qPCR or RNA-seq), analyzing the cDNA to identify reverse transcriptase stops, and mapping the sequence stops onto the sequence or secondary structure of the probed RNA (e.g., an RNA found in a sequence library).

In some cases, contacting a compound disclosed herein with RNA comprises precisely targeting cellular inactivation of an oncogenic non-coding RNA precursor. In some cases, targeting cellular inactivation of an oncogenic non-coding RNA precursor comprises contacting a cell expressing the non-coding RNA precursor with an effective amount of a compound disclosed herein. In some cases, the oncogenic non-coding RNA precursor comprises oncogenic primary microRNA-96 (pri-miR-96). In some cases, the oncogenic non-coding RNA precursor is oncogenic primary microRNA-96 (pri-miR-96).

Also disclosed herein is a method of enhancing the expression of FOXO1 protein in breast cancer cells. In some cases, enhancing the expression of FOXO1 protein in breast cancer cells comprises contacting a cell or cells with an effective amount of a compound disclosed herein.

Also disclosed herein is a method of triggering apoptosis in triple negative breast cancer cells. In some cases, triggering apoptosis in triple negative breast cancer cells comprises contacting the cells with an effective amount of a compound disclosed herein.

Also disclosed herein is a method of treating breast cancer comprising administering to a patient in need thereof an effective amount of a compound disclosed herein. In some cases, the breast cancer is triple negative breast cancer. In some cases, the breast cancer expresses oncongenic primary microRNA-96 (pri-miR-96).

Disclosed herein are methods of methods of mapping an RNA binding site of an RNA-binding small molecule, the binding site being disposed within an RNA sequence library. In some cases, the RNA sequence library comprises a transcriptome. In some cases, the transcriptome is viral, mammalian, or bacterial. In some cases, the transcriptome is viral. In some cases, the transcriptome is mammalian. In some cases, the transcriptome is bacterial. In some cases, the RNA sequence library comprises one or more of synthetic, semi-synthetic, or natural RNA. In some cases, the RNA sequence library comprises synthetic RNA. In some cases, the RNA sequence library comprises semi-synthetic RNA. In some cases, the RNA sequence library comprises natural RNA. In some cases, the RNA sequence library comprises the genome of an RNA virus.

In some cases, the methods disclosed herein are carried out in vitro. In some cases, the methods disclosed herein are carried out in vivo. In some cases, the methods disclosed herein are carried out in living cells. In some cases, the methods disclosed herein are carried out in preclinical animal models. In some cases, the methods disclosed herein are carried out in a human, e.g., in a human patient. In some cases, the cells are virally- or bacterially-infected. In some cases, the cells are virally-infected. In some cases, the cells are bacterially-infected.

Also provided herein are methods wherein a set of RNA sequences and a set of candidate RNA-binding small molecules are assayed in a 2-dimensional parallel assay.

In various embodiments, the method described herein provides a method to identify the cellular targets of small molecules directed at RNA. For example, the ability to cross link and form covalent bonds between an RNA and a small molecule that contains an affinity purification tag (e.g., biotin, as in compound 2) is described. When one of the aforementioned compounds is added to cell culture, the compound enters cells and reacts with the RNA targets of small molecules. The targets are identified by using various methods including streptavidin resin to capture the biotinylated biomolecules. Biomolecules are analyzed via a variety of methods including gel electrophoresis, northern blots, and RNA sequence.

Various embodiments of the disclosed methods and compounds are described below.

1. A method, comprising
(a) contacting RNA and a compound, the compound comprising (i) an RNA-binding module, (ii) an RNA cross-linking module, and (iii) a purification module, such that, upon binding of the RNA-binding module to a binding site on the RNA, the RNA cross-linking module reacts with the RNA to form a covalent bond and thereby a complex of the compound and RNA;
(b) purifying the complex via affinity of the purification module and a complementary immobilized reagent; and
(c); identifying the binding site of the RNA-binding module on the RNA.

2. The method of paragraph 1, wherein identifying comprises
(1) reverse transcribing the RNA from purified complex with a primer to create cDNA;
(2) amplifying the cDNA using a primer set suitable for RT-qPCR or high throughput sequencing (RNA-seq);
(3) analyzing the cDNA by RNA-seq or RT-qPCR to identify the targets, as determined by the enrichment in the purified fractions as compared to starting cell lysate;
(4) analyzing the cDNA to identify reverse transcriptase sequence stops; and
(5) mapping the sequence stops onto the sequence or secondary structure of the RNA of the sequence library.

3. The method of paragraph 1, wherein the identifying comprises
(1) reverse transcribing the RNA from the purified complex with a primer to create cDNA;
(2) amplifying the cDNA with a primer set suitable for RT-qPCR or high-throughput sequencings (RNA-seq); and
(3) analyzing the cDNA to identify reverse transcriptase sequence stops to thereby determine the sequence of the binding site of the RNA-binding module on the RNA.

4. The method of any one of paragraphs 1 to 3, wherein the RNA cross-linking module comprises a N,N-bis(2-chloroethyl)aniline (chlorambucil) group.

5. The method of any one of paragraphs 1 to 4, wherein the purification module comprises a biotin group.

6. The method of any one of paragraphs 1 to 5, wherein the compound is compound 2.

7. The method of any one of paragraphs 1 to 6, wherein the RNA-binding module comprises an N-methyl-piperazinyl-bis-benzimidazole group.

8. The method of any one of paragraphs 1 to 7, wherein the RNA-binding module comprises Targaprimir-96 (compound 1).

9. The method of any one of paragraphs 1 to 8, wherein the RNA comprises an oncogenic non-coding RNA precursor, and the contacting of step (a) comprises contacting a cell expressing the non-coding RNA precursor and the compound.

10. The method of paragraph 9, wherein the oncogenic non-coding RNA precursor comprises oncogenic primary microRNA-96 (pri-miR-96).

11. The method of any one of paragraphs 1 to 10, wherein the RNA comprises a transcriptome.

12. The method of paragraph 11, wherein the transcriptome is viral.

13. The method of paragraph 11, wherein the transcriptome is mammalian.

14. The method of paragraph 11, wherein the transcriptome is bacterial.

15. The method of any one of paragraphs 1 to 14, wherein the RNA comprises one or more of synthetic, semi-synthetic, or natural RNA.

16. The method of any one of paragraphs 1 to 15, wherein the RNA comprises the genome of an RNA virus.

17. The method of any one of paragraphs 1 to 16 carried out in vitro.

18. The method of any one of paragraphs 1 to 17 carried out in living cells.

19. The method of any one of paragraphs 1 to 18 carried out in a preclinical animal model.

20. The method of paragraph 18, wherein the living cells are virally- or bacterially-infected cells.

21. The method of paragraph 1, wherein the method is performed in a parallel array for a plurality of RNA sequences and candidate RNA-binding small modules.

22. A method of enhancing expression of FOXO1 in breast cancer cells, comprising contacting the cells with a compound comprising (i) an RNA-binding module and (ii) an RNA cross-linking module, wherein the RNA-binding module binds to primary microRNA-96 (pri-miR-96) and upon binding of the compound to pri-miR-96 expression of FOXO1 in increased.

23. The method of paragraph 22, wherein the contacting comprises administering the compound to a subject suffering from breast cancer.

24. The method of paragraph 22 or 23, wherein the RNA-binding module comprises Targaprimir-96 (compound 1) and the RNA cross-linking module comprises chlorambucil.

25. A method of triggering apoptosis in triple negative breast cancer cells, comprising contacting the cells with an effective amount of a compound comprising (i) an RNA-binding module and (ii) an RNA cross-linking module, wherein the RNA-binding module binds to a target RNA and upon binding thereby triggers apoptosis in the breast cancer cell.

26. The method of paragraph 25, wherein the contacting comprises administering the compound to a subject suffering from breast cancer.

27. The method of paragraph 25 or 26, wherein the RNA-binding module comprises Targaprimir-96 (compound 1) and the RNA cross-linking module comprises chlorambucil.

28. A method of treating triple negative breast cancer in a patient, comprising administering to the patient an effective amount of a compound comprising (1) (i) an RNA-binding module, (ii) an RNA cross-linking module.

29. The method of paragraph 28, wherein the breast cancer expresses oncogenic primary microRNA-96 (pri-miR-96) and the RNA-binding module binds to pri-miR-96.

30. The method of paragraph 28 or 29, wherein the RNA-binding module comprises Targaprimir-96 and the RNA cross-linking module comprises chlorambucil.

31. A compound comprising (1) an RNA-cross-linking module and (2) a RNA-binding module.

32. The compound of paragraph 31, wherein the RNA cross-linking module comprises an N,N-bis(2-chloroethyl)aniline (chlorambucil) group.

33. The compound of paragraph 31 or 32 wherein the RNA-binding module comprises an N-methyl-piperazinyl-bis-benzimidazole group.

34. The compound of paragraph 33, wherein the RNA-binding module comprises Targaprimir-96.

What is claimed is:

1. A method of mapping an RNA binding site of an RNA-binding small molecule, the binding site being disposed within an RNA sequence library, comprising
contacting the RNA of the sequence library and an effective amount of a compound, the compound comprising a conjugate of the RNA-binding small molecule and an RNA cross-linking module and a purification module, such that the RNA cross-linking module can react with the RNA associated with the RNA binding site of the small molecule; then,
purifying the compound and an RNA target cross-linked by the cross-linking module using affinity of the purification module and a complementary immobilized reagent; then,
reverse transcribe the RNA with a primer to create cDNA from the purified RNA bound and thereby cross-linked to the small molecule; then,
amplify the cDNA using a primer set suitable for RT-qPCR or high throughput sequencing (RNA-seq) then,
analyze the cDNA by RNA-seq or RT-qPCR to identify the target, as determined by the enrichment in the purified fractions; then
analyze the cDNA to identify reverse transcriptase sequence stops; then,
map the sequence stops onto the sequence or secondary structure of the RNA of the sequence library;
to identify the binding sites of the RNA-binding small molecule within the RNA library.

2. The method of claim 1 wherein the RNA cross-linking module comprises a N,N-bis(2-chloroethyl)aniline (chlorambucil) group.

3. The method of claim 1 wherein the purification module comprises a biotin group.

4. The method of claim 1 wherein the compound of claim 1 is of formula 2.

5. The method of claim 1 wherein the compound of claim 1 comprises an N-methyl-piperazinyl-bis-benzimidazole group.

6. The method of claim 1 wherein the compound of claim 1 is Targaprimir-96 (compound 1).

7. The method of claim 1, comprising precisely targeting cellular inactivation of an oncogenic non-coding RNA precursor via cross-linking, comprising contacting a cell expressing the non-coding RNA precursor and an effective amount of the compound of claim 1.

8. The method of claim 7 wherein the oncogenic non-coding RNA precursor comprises oncogenic primary microRNA-96 (pri-miR-96).

9. The method of claim 7 wherein the conjugate is a cross-linking conjugate of Targaprimir-96 and chlorambucil.

10. The method of claim 1, comprising enhancing expression of FOXO1 protein in breast cancer cells, comprising contacting the cells with an effective amount of the compound of claim 1.

11. The method of claim 10 wherein the breast cancer cells are present in a human patient.

12. The method of claim 10 wherein the compound is a covalent conjugate of Targaprimir-96 and chlorambucil.

13. The method of claim 1, comprising triggering apoptosis in triple negative breast cancer cells, comprising contacting the cells with an effective amount of the compound of claim 1.

14. The method of claim 13 wherein the breast cancer cells are present in a human patient.

15. The method of claim 13 wherein the compound is a covalent conjugate of Targaprimir-96 and chlorambucil.

16. The method of claim 1, comprising treating triple negative breast cancer, comprising administering to a patient afflicted therewith an effective dose of the conjugate.

17. The method of claim 16 wherein the breast cancer comprises expression of oncogenic primary microRNA-96 (pri-miR-96).

18. The method of claim 15 wherein the compound is a covalent conjugate of Targaprimir-96 and chlorambucil.

19. The method of claim 1 wherein the RNA sequence library comprises a transcriptome.

20. The method of claim 19 wherein the transcriptome is viral.

21. The method of claim 19 wherein the transcriptome is mammalian.

22. The method of claim 19 wherein the transcriptome is bacterial.

23. The method of claim 1 wherein the RNA sequence library comprises one or more of synthetic, semi-synthetic, or natural RNA.

24. The method of claim 1 wherein the RNA sequence library comprises the genome of an RNA virus.

25. The method of claim 1 carried out in vitro.

26. The method of claim 1 carried out in living cells.

27. The method of claim 1 carried out in preclinical animal models.

28. The method of claim 26 wherein the cells are virally- or bacterially-infected cells.

29. The method of claim 1 wherein a set of RNA sequences and a set of candidate RNA-binding small molecules are assayed in a 2-dimensional parallel array.

30. A conjugate of an RNA-cross-linking moiety and a RNA-binding small molecule.

31. The conjugate of claim 30 wherein the RNA-cross-linking moiety comprises an N,N-bis(2-chloroethyl)aniline (chlorambucil) group.

32. The conjugate of claim 30 wherein the RNA-binding small molecule comprises an N-methyl-piperazinyl-bis-benzimidazole group.

33. The conjugate of claim 30, wherein the RNA-binding small molecule is Targaprimir-96.
